(19) United States
(12) Patent Application Publication
(10) Pub. No.: US 2018/0177588 A1
(43) Pub. Date: Jun. 28, 2018

(54) SCLERAL PROSTHESIS HAVING SERRATED OR OTHER SURFACE(S) FOR RETENTION IN SCLERAL TISSUE FOR TREATING PRESBYOPIA AND OTHER EYE DISORDERS

(71) Applicant: Refocus Group, Inc., Dallas, TX (US)

(72) Inventor: Kevin J. Cady, Saint Charles, IL (US)

(21) Appl. No.: 15/849,576

(22) Filed: Dec. 20, 2017

Related U.S. Application Data
(60) Provisional application No. 62/438,700, filed on Dec. 23, 2016.

(51) Int. Cl. A61F 2/14; A61F 9/00

(52) U.S. Cl. CPC A61F 2/147; A61F 9/00781; A61F 9/0017

(57) ABSTRACT

A scleral prosthesis includes an elongated body configured to be implanted into scleral tissue of an eye. The elongated body includes opposing first and second free ends and at least one serrated or textured surface between the first and second free ends. The at least one serrated or textured surface is configured to allow sliding of the elongated body within the scleral tissue in a first direction and to resist sliding of the elongated body within the scleral tissue in a second direction opposite to the first direction.

### CROSS-REFERENCE TO RELATED APPLICATION AND PRIORITY CLAIM

This application claims priority under 35 U.S.C. § 119(e) to U.S. Provisional Patent Application No. 62/438,700 filed on Dec. 23, 2016. This provisional application is hereby incorporated by reference in its entirety.

### TECHNICAL FIELD

This disclosure is generally directed to ocular implants. More specifically, this disclosure is directed to a scleral prosthesis having one or more serrated or other surfaces for retention in scleral tissue for treating presbyopia and other eye disorders.

### BACKGROUND

Presbyopia is a condition that causes a person to slowly lose the ability to focus his or her eyes on objects close to the person. As a result, the person comes to need glasses for tasks requiring near vision, such as reading. Presbyopia is observed in many people over the age of forty. While the conventional view was that presbyopia could not be treated, recent advances have shown that presbyopia can be treated by implanting scleral prostheses within tunnels formed through the sclera of a patient's eye.

### SUMMARY

This disclosure provides a scleral prosthesis having one or more serrated or other surfaces for retention in scleral tissue for treating presbyopia and other eye disorders.

In a first embodiment, a scleral prosthesis includes an elongated body configured to be implanted into scleral tissue of an eye. The elongated body includes opposing first and second free ends and at least one serrated or textured surface between the first and second free ends. The at least one serrated or textured surface is configured to allow sliding of the elongated body within the scleral tissue in a first direction and to resist sliding of the elongated body within the scleral tissue in a second direction opposite to the first direction.

In particular embodiments, any single one or any combination of the following features could be used with the first embodiment. The elongated body could include a top surface extending from the first free end to the second free end and a bottom surface extending from the first free end to the second free end. The at least one serrated or textured surface could include the bottom surface. At least part of the top surface could be convex, and at least part of the bottom surface could be planar. The scleral prosthesis could further include an opening through the first end, where the opening is configured to be grasped by a surgical tool. The elongated body could be linear and could have a substantially equal width along its length. The at least one serrated or textured surface could include multiple grooves forming multiple angled projections. The grooves and the angled projections could extend substantially across an entire width of the elongated body. The elongated body could be configured to be implanted lengthwise into the scleral tissue of the eye. A thickness of the elongated body could taper towards the first and second ends.

In a second embodiment, a method includes forming an elongated body of a scleral prosthesis that is configured to be implanted into scleral tissue of an eye. The elongated body includes opposing first and second free ends and at least one serrated or textured surface between the first and second free ends. The at least one serrated or textured surface is configured to allow sliding of the elongated body within the scleral tissue in a first direction and to resist sliding of the elongated body within the scleral tissue in a second direction opposite to the first direction.

In particular embodiments, any single one or any combination of the following features could be used with the second embodiment. The elongated body could include a top surface extending from the first free end to the second free end and a bottom surface extending from the first free end to the second free end. The at least one serrated or textured surface could include the bottom surface. At least part of the top surface could be convex, and at least part of the bottom surface could be planar. The method could further include forming an opening through the first end, where the opening is configured to be grasped by a surgical tool. The elongated body could be linear and could have a substantially equal width along its length. The at least one serrated or textured surface could include multiple grooves forming multiple angled projections. The grooves and the angled projections could extend substantially across an entire width of the elongated body. The elongated body could be configured to be implanted lengthwise into the scleral tissue of the eye. A thickness of the elongated body could taper towards the first and second ends.

In a third embodiment, a scleral prosthesis includes an elongated body configured to be implanted into scleral tissue of an eye. The elongated body includes opposing first and second free ends and at least one serrated or textured surface between the first and second free ends. The scleral prosthesis also includes an opening through the first end, where the opening is configured to be grasped by a surgical tool. The at least one serrated or textured surface is configured to allow sliding of the elongated body within the scleral tissue in a first direction and to resist sliding of the elongated body within the scleral tissue in a second direction opposite to the first direction. The elongated body includes a top surface extending from the first free end to the second free end and a bottom surface extending from the first free end to the second free end. At least part of the top surface is convex, and at least part of the bottom surface is planar. The at least one serrated or textured surface includes multiple grooves forming multiple angled projections. A thickness of the elongated body tapers towards the first and second ends.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawing, in which:

FIGS. 1 through 4 illustrate an example scleral prosthesis in accordance with this disclosure;

FIG. 5 illustrates an example cross-section of the scleral prosthesis shown in FIGS. 1 through 4 in accordance with this disclosure;

FIG. 6 illustrates an enlarged portion of the example cross-section of the scleral prosthesis shown in FIG. 5 in accordance with this disclosure;

FIG. 7 illustrates an example method for forming a scleral prosthesis having one or more serrated or other surfaces for retention in scleral tissue in accordance with this disclosure; and

FIG. 8 illustrates an example method for using a scleral prosthesis having one or more serrated or other surfaces for retention in scleral tissue in accordance with this disclosure.

### DETAILED DESCRIPTION

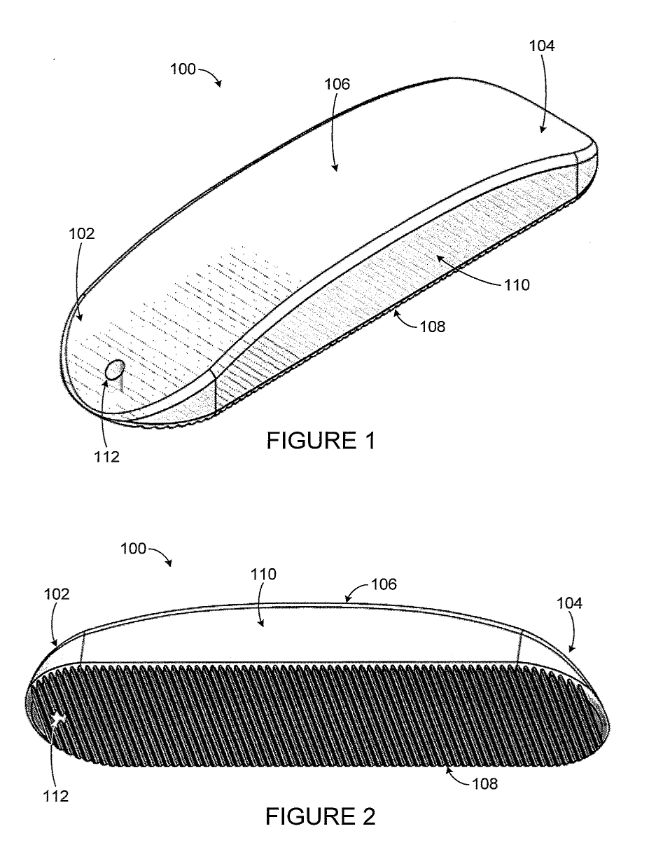

FIGS. 1 through 8, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

FIGS. 1 through 4 illustrate an example scleral prosthesis 100 in accordance with this disclosure. In particular, FIG. 1 illustrates a top perspective view of the scleral prosthesis 100, and FIG. 2 illustrates a bottom perspective view of the scleral prosthesis 100. Also, FIG. 3 illustrates atop view of the scleral prosthesis 100, and FIG. 4 illustrates an end view of the scleral prosthesis 100. The embodiment of the scleral prosthesis 100 shown in FIGS. 1 through 4 is for illustration only. Other embodiments of the scleral prosthesis 100 could be used without departing from the scope of this disclosure.

As shown in FIGS. 1 through 4, the scleral prosthesis 100 is formed by an elongated body that is configured to be implanted into scleral tissue of a patient's eye. The elongated body includes two opposing ends 102 and 104, a top surface 106 and a bottom surface 108 extending between the ends 102 and 104, and two opposing sides 110 extending between the ends 102 and 104. The elongated body of the scleral prosthesis 100 is shown here as being a single integral structure. However, the elongated body could be formed from multiple pieces that are coupled together to form the scleral prosthesis 100. If implemented using multiple pieces, the multiple pieces could be coupled together using mechanical connectors, using magnetic connectors, or in any other suitable manner.

In this example, the scleral prosthesis 100 has a generally linear shape, meaning the elongated body is generally straight and has a substantially equal width along its entire length. Note, however, that the elongated body could have any other suitable shape, such as an hourglass shape, an “I” shape, an “X” shape, or a “T” shape. In some cases, one or both ends 102 and 104 of the elongated body could be wider than a remaining portion of the elongated body between the ends 102 and 104. One or both ends 102 and 104 of the elongated body could also or alternatively be split, meaning that one or both ends 102 and 104 each includes divided portions of the elongated body that are separated by empty space to form that end. The divided portions could be pushed towards each other, such as to reduce the width of at least one end of the elongated body during implantation, and the divided portions could expand again once released. Optionally, an insert could be placed into the empty space to maintain separation of the divided portions of the elongated body, such as after implantation.

In the example shown in FIGS. 1 through 4, the top surface 106 is generally convex between the ends 102 and 104, and the bottom surface 108 is generally planar between the ends 102 and 104. This means the elongated body has a thickness (measured top to bottom) that tapers towards its ends 102 and 104. However, other shapes could be used for the top and bottom surfaces 106 and 108. For example, the top surface 106 could be convex along most of the scleral prosthesis' length and have small concave portions at the ends 102 and 104 of the body (similar to that shown in U.S. Pat. No. 7,416,560, which is hereby incorporated by reference in its entirety). As another example, the bottom surface 108 could be partially or fully concave or partially or fully convex along the scleral prosthesis' length.

The sides 110 generally extend between the top and bottom surfaces 106 and 108 of the elongated body. In the example shown in FIGS. 1 through 4, the sides 110 are generally flat and extend straight between the ends 102 and 104 of the body and between the top and bottom surfaces 106 and 108 of the body. However, other shapes could be used for the sides 110. For example, each of the sides 110 could be partially or fully convex or partially or fully concave along the scleral prosthesis' length. Also, the top and bottom surfaces 106 and 108 could join in a manner other than what is shown here, which can alter the shape and size of the sides 110. In some instances, the sides 110 may be very small or non-existent depending on how the top and bottom surfaces 106 and 108 meet.

Various prior scleral prostheses (such as those disclosed in U.S. Pat. No. 9,498,324, which is hereby incorporated by reference in its entirety) were often described as being inserted into scleral tunnels of patients' eyes. A scleral tunnel denotes a passageway that both extends into and out of scleral tissue, meaning there are two incisions on the surface of a patient's eye for each tunnel. Also, each of those scleral prostheses was often described as having its two opposing ends remain outside a scleral tunnel after implantation. Thus, one end of those scleral prostheses could be inserted through one incision on the surface of a patient's eye, travel through the scleral tunnel, and exit the patient's eye through the other incision on the surface of the patient's eye. While effective, this often requires the formation of multiple linked incisions per tunnel or the formation of a long single incision per tunnel in a patient's eye.

Rather than being inserted completely through a scleral tunnel, the scleral prosthesis 100 could be inserted into a scleral “cave” or “pocket,” which refers to an incision made into scleral tissue of an eye that ends within the scleral tissue and does not exit from the scleral tissue. As a result, there may be only a single incision on the surface of a patient's eye for each scleral cave, and that single incision could be smaller compared to an incision needed to form a scleral tunnel. Most or all of the scleral prosthesis 100 could then be inserted into a scleral cave in order to treat presbyopia or other eye disorders. When implanted in this manner, the entire scleral prosthesis 100 may be hidden within the patient's scleral tissue, or only a small portion (such as a single end) of the scleral prosthesis 100 may be visible. This can help to increase the cosmetic appeal of a medical treatment that uses the scleral prosthesis 100. Also, a scleral cave can be formed using a trans-conjunctival surgical procedure, meaning an incision is made through the conjunctiva of a patient's eye but not completely through the sclera of the patient's eye (as opposed to a trans-scleral surgical procedure that forms an incision through the sclera of the patient's eye). A trans-conjunctival surgical procedure could be simpler and faster than a trans-scleral surgical procedure and result in fewer injuries that require healing while reducing surgical risks.

Moreover, as described below, at least one surface of the scleral prosthesis 100 can be serrated or otherwise textured. The serrated or other textured surface(s) of the scleral prosthesis 100 can permit the scleral prosthesis 100 to slide in one direction and can resist movement of the scleral prosthesis 100 in the opposite direction. During implantation, one end 102 of the scleral prosthesis 100 can be pushed into a scleral cave, followed by the remainder of the scleral prosthesis 100. The serrated or other textured surface(s) of the scleral prosthesis 100 can allow this movement of the scleral prosthesis 100 into the scleral cave. However, the serrated or other textured surface(s) of the scleral prosthesis 100 can prevent the backward movement of the scleral prosthesis 100 out of the scleral cave. This helps to secure the scleral prosthesis 100 in a desired position within the patient's eye.

In addition, the scleral prosthesis 100 can include an opening 112 at one or both ends 102 and 104 of the scleral prosthesis 100. After implantation in a patient's eye, if the scleral prosthesis 100 needs to be removed, an incision can be made through the conjunctiva and sclera of the patient's eye to expose one end 102 or 104 of the scleral prosthesis 100. A tool could be inserted into the opening 112 by a surgeon or other personnel, and the scleral prosthesis 100 can be pulled or pushed to remove the scleral prosthesis 100 from the scleral cave. The serrated or other textured surface(s) of the scleral prosthesis 100 can allow this movement out of the scleral cave. Note that while a single opening 112 on one end 102 of the scleral prosthesis 100 is shown here, one or more openings 112 could exist at one or both ends 102 and 104 of the scleral prosthesis 100. Multiple openings 112 could be useful, for instance, so that the scleral prosthesis 100 can be removed using any exposed opening 112.

The scleral cave in which a scleral prosthesis 100 is implanted can be formed near the ciliary body of a patient's eye. Once implanted in a scleral cave, the scleral prosthesis 100 helps to, for example, increase the amplitude of accommodation of the patient's eye, which means that the scleral prosthesis 100 can help improve the ability of the patient's eye to focus on nearby objects. The scleral prosthesis 100 can therefore help to treat or reverse presbyopia in the patient. The scleral prosthesis 100 could also help to treat other eye conditions, such as glaucoma, ocular hypertension, elevated intraocular pressure, or other eye disorders. In some embodiments, multiple scleral prostheses 100 are implanted in a patient's eye, such as when four scleral prostheses 100 are implanted in four quadrants of the patient's eye (like at 45°, 135°, 225°, and 315°). Also, the ends of the scleral prostheses 100 are “free,” meaning the ends 102 and 104 of one scleral prosthesis 100 are not attached to the ends 102 and 104 of other scleral prostheses 100.

The scleral cave in which a scleral prosthesis 100 is implanted can also be formed in any suitable manner. For example, various surgical tools for forming scleral tunnels are disclosed in U.S. Pat. No. 7,189,248; U.S. Pat. No. 8,083,759; and U.S. Pat. No. 8,597,318 (which are hereby incorporated by reference in their entirety). Any of these surgical tools could be configured to form a scleral cave, such as by partially rotating or otherwise moving a curved blade or other surgical blade so that the tip of the blade enters into the patient's scleral tissue but does not travel completely through the patient's scleral tissue and form a tunnel. The surgical blade would therefore form a scleral cave based on a single incision in the patient's eye, rather than a scleral tunnel. Also, the surgical tool used to form the scleral caves could be used in conjunction with a docking station or ocular fixation device, such as those disclosed in U.S. Pat. No. 8,709,029 and U.S. Patent Publication No. 2012/0226107 (which are hereby incorporated by reference in their entirety).

The scleral prosthesis 100 could have any suitable size, shape, and dimensions. In some embodiments, the scleral prosthesis 100 could have a length of about 4.5 millimeters and a width of about 1.25 millimeters, and the opening 112 could have a diameter of about 0.125 millimeters. However, other dimensions could be used. Also, scleral prostheses 100 of different sizes could be provided, such as for use with eyes of different sizes or for different medical treatments or surgical procedures. Further, the scleral prosthesis 100 could be formed from any suitable material(s). For example, the scleral prosthesis 100 may be formed using polymethyl methacrylate (“PMMA”), polyether-ether ketone (“PEEK”), or other suitable material(s). In addition, the scleral prosthesis 100 could be formed in any suitable manner, such as via injection molding, machining, or additive manufacturing (such as “3D printing”).

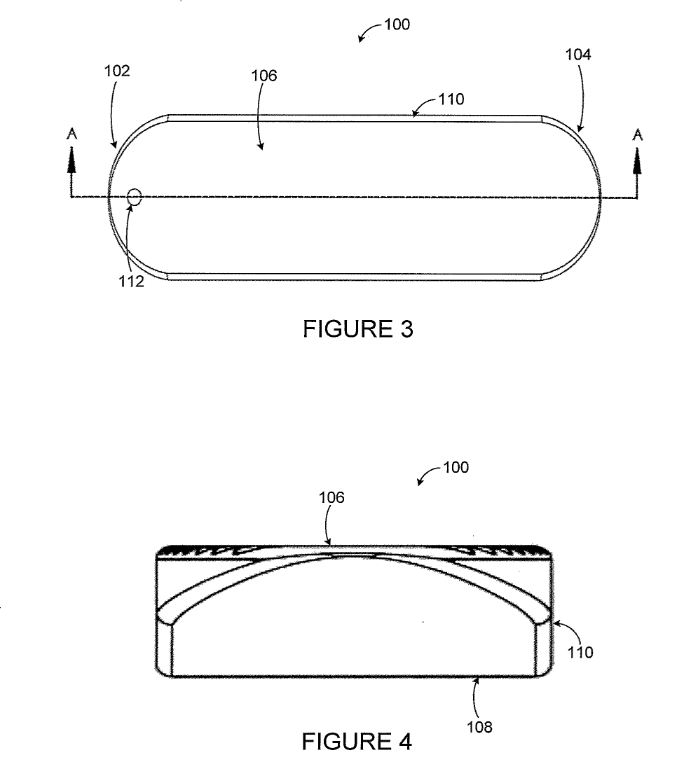

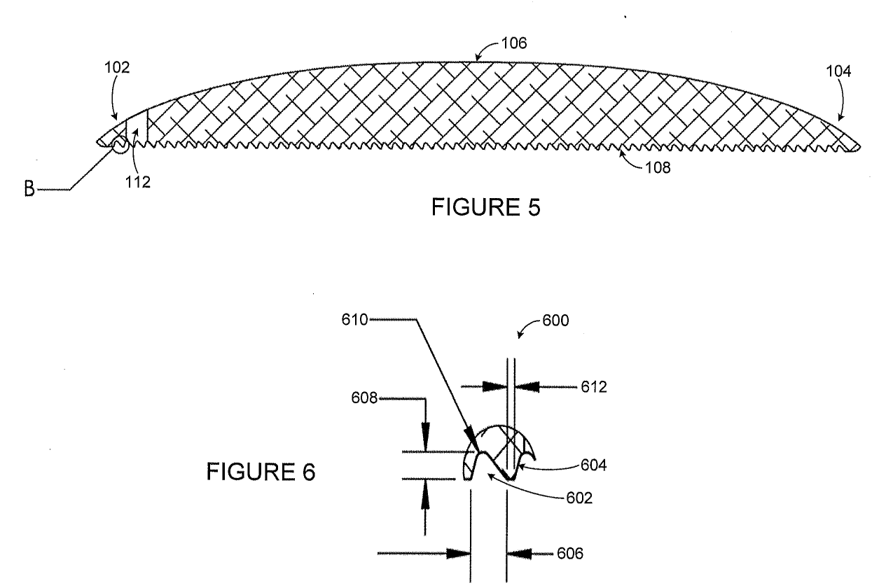

FIG. 5 illustrates an example cross-section of the scleral prosthesis 100 shown in FIGS. 1 through 4 in accordance with this disclosure. FIG. 6 illustrates an enlarged portion 600 of the example cross-section of the scleral prosthesis 100 shown in FIG. 5 in accordance with this disclosure. In particular, FIG. 5 illustrates the cross-section of the scleral prosthesis 100 along lines A-A in FIG. 3, and FIG. 6 illustrates an enlarged view of the cross-section of the scleral prosthesis 100 in the circled portion B of FIG. 5.

A serrated or other textured bottom surface of the scleral prosthesis 100 is shown in FIG. 5. As shown more clearly in FIG. 6, the textured surface is formed by grooves 602 in the surface, which leads to the creation of angled projections like teeth 604. Since the teeth 604 are angled, the teeth 604 can allow movement of the scleral prosthesis 100 in one direction (right to left in FIG. 5) while resisting movement of the scleral prosthesis 100 in the opposite direction (left to right in FIG. 5). Note, however, that other suitable serrations or other texturing could be used to support movement of the scleral prosthesis 100 in one or more selected directions while resisting movement of the scleral prosthesis 100 in one or more other directions.

Any suitable dimensions could be used with the serrations or other texturing. In some embodiments, each groove 602 could have a width 606 of about 0.05 millimeters and a height 608 of about 0.031 millimeters, and a top of each groove 602 could have a radius of curvature 610 of about 0.01 millimeters. There could also be a separation 612 of about 0.01 millimeters between adjacent grooves 602. Note, however, that this shape and these dimensions are examples only and that other shapes and dimensions could be used.

The ability to restrict movement in one or more directions can vary based on the design of the serrations or other texturing. For example, in FIGS. 5 and 6, the ability to restrict movement left to right could be based on the angling of the teeth 604 towards the right. The teeth 604 could therefore dig into ocular tissue when moving left to right in FIGS. 5 and 6, but the teeth 604 could slide over ocular tissue more easily when moving right to left in FIGS. 5 and 6. As another example, ocular tissue could grow partially or completely into the grooves 602, and the teeth 604 could push against this ocular tissue when moving left to right in FIGS. 5 and 6 (but slide more easily over this tissue when moving right to left in FIGS. 5 and 6).

In the example of the scleral prosthesis 100 shown in FIGS. 1 through 6, the textured surface is formed using grooves 602 that extend completely across the width of the scleral prosthesis 100, which means that the teeth 604 also extend completely across the width of the scleral prosthesis 100. However, this need not be the case. For instance, each groove 602 could extend partially across the width of the scleral prosthesis 100. Different areas of the textured surface(s) of the scleral prosthesis 100 could have different textures, or some areas of the textured surface(s) of the scleral prosthesis 100 could have texturing while other areas of the textured surface(s) of the scleral prosthesis 100 could lack texturing.

Although FIGS. 1 through 6 illustrate one example of a scleral prosthesis 100 and related details, various changes may be made to FIGS. 1 through 6. For example, the sizes, shapes, and relative dimensions of the features of the scleral prosthesis 100 could vary as needed or desired. As particular examples, the sides 110 of the scleral prosthesis 100 could be slanted, or various surfaces of the scleral prosthesis 100 could have different shapes. Also, the use of an opening 112 as a mechanism for grasping the scleral prosthesis 100 is one example type of structure supporting manipulation of the scleral prosthesis 100, although various other structures could also be used as a mechanism for grasping or moving the scleral prosthesis 100.

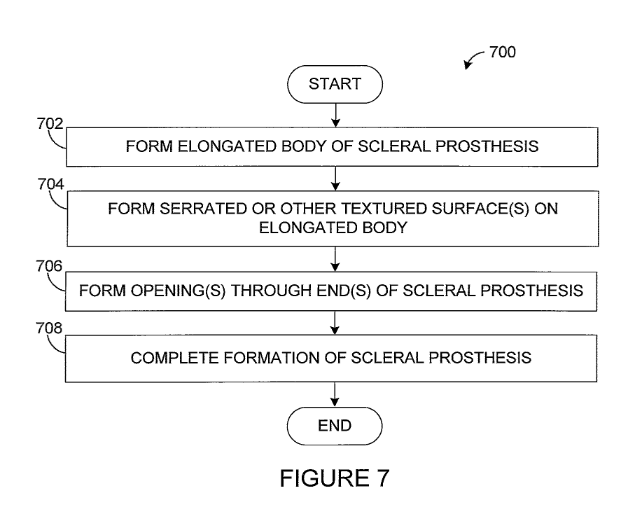

FIG. 7 illustrates an example method 700 for forming a scleral prosthesis having one or more serrated or other surfaces for retention in scleral tissue in accordance with this disclosure. For ease of explanation, the method 700 is described with respect to the scleral prosthesis 100 of FIGS. 1 through 6. However, as noted above, various modifications could be made to the scleral prosthesis 100, which could affect how the scleral prosthesis 100 is formed.

As shown in FIG. 7, an elongated body of a scleral prosthesis is formed at step 702, and at least one serrated or other textured surface is formed on the elongated body at step 704. This could include, for example, forming an elongated body having two opposing ends 102 and 104 and top and bottom surfaces 106 and 108. This could also include forming grooves 602 across the bottom surface 108 or other or additional surface(s) of the elongated body to form serrations. As particular examples, this could include forming the texturing on the elongated body during or after formation of the elongated body. As noted above, however, any suitable texturing could be used, and the texturing could be used on one, some, or all surfaces of the elongated body.

One or more openings are formed through one or more ends of the elongated body at step 706. This could include, for example, forming one or more openings 112 in one end 102 of the elongated body or forming openings 112 in both ends 102 and 104 of the elongated body. As particular examples, this could include drilling through the elongated body to form the opening(s) 112 or forming the opening(s) 112 while forming the elongated body. Formation of the scleral prosthesis is completed at step 708. This could include, for example, polishing the surfaces of the elongated body, sterilizing the elongated body, or performing any other or additional actions to form a completed scleral prosthesis.

Although FIG. 7 illustrates one example of a method 700 for forming a scleral prosthesis having one or more serrated or other surfaces for retention in scleral tissue, various changes may be made to FIG. 7. For example, while shown as a series of steps, various steps in FIG. 7 could overlap, occur in parallel, occur in a different order, or occur any number of times. As a particular example, some or all of the steps 702-706 could overlap or occur at the same time, such as when most or all features of the elongated body are formed using injection molding or other fabrication technique that involves the formation of these features at substantially the same time.

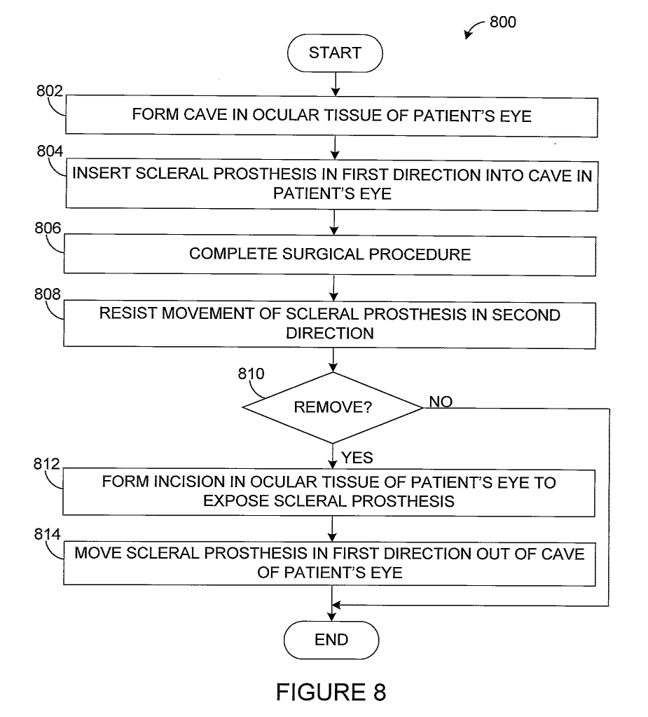

FIG. 8 illustrates an example method 800 for using a scleral prosthesis having one or more serrated or other surfaces for retention in scleral tissue in accordance with this disclosure. For ease of explanation, the method 800 is described with respect to the scleral prosthesis 100 of FIGS. 1 through 6. However, as noted above, various modifications could be made to the scleral prosthesis 100, which could affect how the scleral prosthesis 100 is used.

As shown in FIG. 8, a scleral cave is formed in a patient's ocular tissue at step 802. This could include, for example, forming a single incision in the sclera of a patient's eye, where the incision extends into the patient's sclera but not out of the patient's sclera. The scleral cave could be formed manually or using an automated tool. In some embodiments, the incision could be formed as part of a trans-conjunctival surgical procedure.

A scleral prosthesis is inserted in a first direction into the scleral cave in the patient's eye at step 804. This could include, for example, inserting the elongated body of the scleral prosthesis 100 lengthwise into the scleral cave. The first direction denotes a direction in which the serrations or other texturing of the scleral prosthesis 100 permits or allows sliding or other movement of the scleral prosthesis 100. A surgical procedure is completed at step 806. This could include, for example, performing any other actions to complete a surgical procedure for treating presbyopia or other eye disorders. Once implanted in this manner, the serrations or other texturing of the scleral prosthesis resists movement of the scleral prosthesis in a second direction opposite to the first direction at step 808. The second direction denotes a direction in which the serrations or other texturing of the scleral prosthesis 100 restricts sliding or other movement of the scleral prosthesis 100.

It may become necessary or desirable at some point to remove the scleral prosthesis at step 810. This may occur, for example, if a surgical complication or other complication associated with the scleral prosthesis 100 arises. This may also occur if there is a need to replace the scleral prosthesis 100 with another scleral prosthesis. If this condition arises, an incision is formed in the ocular tissue of the patient's eye to expose at least part of the scleral prosthesis at step 812. This could include, for example, forming an incision near the end 102 of the scleral prosthesis 100 and revealing an opening 112 of the scleral prosthesis 100. The scleral prosthesis is moved in the first direction out of the scleral cave in the patient's eye at step 814. This could include, for example, a surgeon or other personnel grabbing the opening 112 of the scleral prosthesis 100 using a surgical tool and pulling the scleral prosthesis 100 out of the scleral cave.

Although FIG. 8 illustrates one example of a method 800 for using a scleral prosthesis having one or more serrated or other surfaces for retention in scleral tissue, various changes may be made to FIG. 8. For example, while shown as a series of steps, various steps in FIG. 8 could overlap, occur in parallel, occur in a different order, or occur any number of times. As a particular example, multiple scleral prostheses could be inserted into and possibly removed from the patient's eye, and one or both eyes of the patient could undergo treatment as described above.

It may be advantageous to set forth definitions of certain words and phrases used throughout this patent document. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in this patent document should not be read as implying that any particular element, step, or function is an essential or critical element that must be included in the claim scope. Also, none of the claims is intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim is understood and intended to refer to structures known to those skilled in the relevant art, as further modified or enhanced by the features of the claims themselves, and is not intended to invoke 35 U.S.C. § 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A scleral prosthesis comprising:
an elongated body configured to be implanted into scleral tissue of an eye, the elongated body comprising opposing first and second free ends and at least one serrated or textured surface between the first and second free ends;
wherein the at least one serrated or textured surface is configured to allow sliding of the elongated body within the scleral tissue in a first direction and to resist sliding of the elongated body within the scleral tissue in a second direction opposite to the first direction.

2. The scleral prosthesis of claim 1, wherein the elongated body comprises:
a top surface extending from the first free end to the second free end; and
a bottom surface extending from the first free end to the second free end.

3. The scleral prosthesis of claim 2, wherein the at least one serrated or textured surface comprises the bottom surface.

4. The scleral prosthesis of claim 2, wherein:
at least part of the top surface is convex; and
at least part of the bottom surface is planar.

5. The scleral prosthesis of claim 1, further comprising:
an opening through the first end, the opening configured to be grasped by a surgical tool.

6. The scleral prosthesis of claim 1, wherein the elongated body is linear and has a substantially equal width along its length.

7. The scleral prosthesis of claim 1, wherein the at least one serrated or textured surface comprises multiple grooves forming multiple angled projections.

8. The scleral prosthesis of claim 7, wherein the grooves and the angled projections extend substantially across an entire width of the elongated body.

9. The scleral prosthesis of claim 1, wherein the elongated body is configured to be implanted lengthwise into the scleral tissue of the eye.

10. The scleral prosthesis of claim 1, wherein a thickness of the elongated body tapers towards the first and second ends.

11. A method comprising:
forming an elongated body of a scleral prosthesis that is configured to be implanted into scleral tissue of an eye, the elongated body comprising opposing first and second free ends and at least one serrated or textured surface between the first and second free ends;
wherein the at least one serrated or textured surface is configured to allow sliding of the elongated body within the scleral tissue in a first direction and to resist sliding of the elongated body within the scleral tissue in a second direction opposite to the first direction.

12. The method of claim 11, wherein the elongated body comprises:
a top surface extending from the first free end to the second free end; and
a bottom surface extending from the first free end to the second free end.

13. The method of claim 12, wherein the at least one serrated or textured surface comprises the bottom surface.

14. The method of claim 12, wherein:
at least part of the top surface is convex; and
at least part of the bottom surface is planar.

15. The method of claim 11, further comprising:
forming an opening through the first end, the opening configured to be grasped by a surgical tool.

16. The method of claim 11, wherein the elongated body is linear and has a substantially equal width along its length.

17. The method of claim 11, wherein the at least one serrated or textured surface comprises multiple grooves forming multiple angled projections.

18. The method of claim 11, wherein the elongated body is configured to be implanted lengthwise into the scleral tissue of the eye.

19. The method of claim 11, wherein a thickness of the elongated body tapers towards the first and second ends.

20. A scleral prosthesis comprising:
an elongated body configured to be implanted into scleral tissue of an eye, the elongated body comprising opposing first and second free ends and at least one serrated or textured surface between the first and second free ends; and
an opening through the first end, the opening configured to be grasped by a surgical tool;
wherein the at least one serrated or textured surface is configured to allow sliding of the elongated body within the scleral tissue in a first direction and to resist sliding of the elongated body within the scleral tissue in a second direction opposite to the first direction;
wherein the elongated body comprises a top surface extending from the first free end to the second free end and a bottom surface extending from the first free end to the second free end, at least part of the top surface being convex, at least part of the bottom surface being planar;
wherein the at least one serrated or textured surface comprises multiple grooves forming multiple angled projections; and
wherein a thickness of the elongated body tapers towards the first and second ends.
